(19) United States
(12) Patent Application Publication
(10) Pub. No.: US 2005/0124497 A1
(43) Pub. Date: Jun. 9, 2005

(54) Delta1-pyrrolines and their use as pest control agents

(76) Inventors: Martin Fusslein, Dusseldorf (DE); Thomas Seitz, Langenfeld (DE); Johannes Rudolf Jansen, Monheim (DE); Udo Kraatz, Leverkusen (DE); Christoph Erdelen, Leichlingen (DE); Angelika Lubos-Erdelen, Leichlingen (DE); Andreas Turberg, Haan (DE); Olaf Hansen, Leichlingen (DE)

Correspondence Address: BAYER CROPSCIENCE LP;Patent Department, 100 BAYER ROAD, PITTSBURGH, PA, 15205-9741, US

(21) Appl. No.: 10/502,665

(22) PCT Filed: Jan. 21, 2003
(86) PCT No.: PCT/EP03/00516

(30) Foreign Application Priority Data
Feb. 1, 2002 (DE) 102 04 115.6

(51) Int. Cl. C07D 491/02; C07D 498/02; A01N 43/40; A01N 43/42

(52) U.S. Cl. 504/246; 504/267; 504/270; 514/301; 514/302; 514/367; 514/375; 546/114; 546/115; 548/152; 548/217

(57) ABSTRACT

The present invention related to novel Δ¹-pyrrolines of the formula (I) 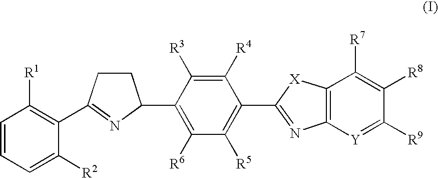 in which
R¹, R², R³, R⁴, R⁵, R⁶ , X, Y, R⁷, R⁸ and R⁹ are as defined in the description disclosure. to a plurality of precesses for preparing these substances and their use for controlling pests, and to novel intermediates and processes for their preparation.

The present invention relates to novel Δ¹-pyrrolines, to a plurality of processes for their preparation and to their use as pesticides.

It is already known that numerous Δ¹-pyrrolines have insecticidal properties (cf. WO 00/21958, WO 99/59968, WO 99/59967 and WO 98/22438). The activity of these substances is good; however, in some cases it is unsatisfactory.

This invention now provides novel Δ¹-pyrrolines of the formula (I) 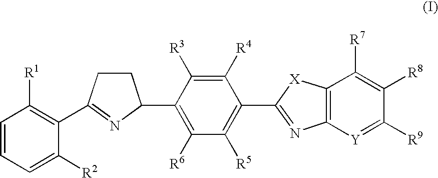
in which
R¹ represents halogen or methyl,
R² represents hydrogen or halogen,
R³, R⁴, R⁵ and R⁶ independently of one another represent hydrogen, halogen, C₁-C₄-alkyl or C₁-C₄-alkoxy,
X represents O (oxygen) or S (sulphur),
Y represents C¹⁰ or N (nitrogen),
R⁷, R⁸, R⁹ and R¹⁰ independently of one another represent hydrogen, halogen, nitro, C₁-C₄-alkyl, C₁-C₄-haloalkyl, C₁-C₄-haloalkoxy, C₁-C₄-haloalkylthio, C₁-C₄-alkoxycarbonyl, phenyl, —(CH)ₘ—SO₂R¹¹ or —SO₂NR¹²R¹³, or either R⁷ and R⁸ or R⁸ and R⁹ or R⁹ and R¹⁰ together form a further saturated or unsaturated 5- or 6-membered ring which again may contain one or two heteroatom groupings from the group consisting of N, O, S and SO₂,
m represents 0 or 1,
R¹¹ represents C₁-C₄-alkyl or morpholino,
R¹² represents C₁-C₄-alkyl or represents phenyl which is optionally mono- to tetrasubstituted by identical or different substituents from the group consisting of halogen, C₁-C₄-alkyl and C₁-C₄-alkoxy,
R¹³ represents hydrogen or C₁-C₄-alkyl.

Depending on the type and number of substituents, the compounds of the formula (I) may be present as geometrical and/or optical isomers or regioisomers or as mixtures of these isomers in varying composition. What is claimed by the invention are both the pure isomers and the isomer mixtures.

Furthermore, it has been found that the Δ¹-pyrrolines of the formula (I) can be prepared by
A) reacting compounds of the formula (II) 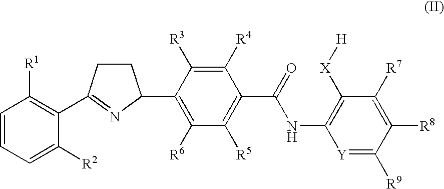
   in which
R¹, R², R³, R⁴, R⁵, R⁶, X, Y, R⁷, R⁸ and R⁹ are as defined above, with p-toluenesulphonic acid, if appropriate in the presence of a diluent.

Finally, it has been found that the compounds of the formula (1) according to the invention have very good insecticidal properties and can be used both in crop protection and in the protection of materials for controlling unwanted pests, such as insects.

The formula (I) provides a general definition of the Δ¹-pyrrolines according to the invention.

Preference is given to Δ¹-pyrrolines of the formula (I) in which
R¹ represents fluorine, chlorine, bromine or methyl,
R² represents hydrogen, fluorine or chlorine,
R³, R⁴, R⁵ and R⁶ independently of one another represent hydrogen, fluorine, chlorine, bromine, C₁-C₄-alkyl or C₁-C₄-alkoxy,
X represents O (oxygen) or S (sulphur),
Y represents CR¹⁰ or N (nitrogen),
R⁷, R⁸, R⁹ and R¹⁰ independently of one another represent hydrogen, fluorine, chlorine, bromine, nitro, C₁-C₄-alkyl; C₁-C₄-haloalkyl, C₁-C₄-haloalkoxy, C₁-C₄-haloalkylthio having in each case 1 to 9 fluorine, chlorine and/or bromine atoms; C₁-C₄-alkoxycarbonyl, phenyl, —(CH₂)ₘ—SO₂R¹¹ or —SO₂NR¹²R¹³, or either R⁷ and R⁸ or R⁸ and R⁹ or R⁹ and R¹⁰ together form a further saturated or unsaturated 5- or 6-membered ring which again may contain one or two heteroatom groupings from the group consisting of N, O, S and SO₂,
m represents 0 or 1,
R¹¹ represents C₁-C₄-alkyl or-morpholino,
R¹² represents C₁-C₄-alkyl or represents phenyl which is optionally mono- to tetrasubstituted by identical or different substituents from the group consisting of fluorine, chlorine, bromine, C₁-C₄-alkyl and C₁-C₄-alkoxy,
R¹³ represents hydrogen or C₁-C₄-alkyl.

Particular preference is given to Δ¹-pyrrolines of the formula (I) in which
R¹ represents fluorine, chlorine or methyl,
R² represents hydrogen, fluorine or chlorine,
R³ and R⁶ independently of one another represent hydrogen, fluorine, chlorine, methyl, ethyl, n-propyl, i-propyl, n-butyl, i-butyl, s-butyl, t-butyl, methoxy or ethoxy,
R⁴ and R⁵ each represent hydrogen,
x represents O (oxygen) or S (sulphur),
Y represents CR¹⁰ or N (nitrogen),
R⁷, R⁸, R⁹ and R¹⁰ independently of one another represent hydrogen, fluorine, chlorine, bromine, nitro, methyl, ethyl, n-propyl, i-propyl, n-butyl, i-butyl, s-butyl, t-butyl; C₁-C₄-haloalkyl, C₁-C₄-haloalkoxy, C₁-C₄-haloalkylthio having in each case 1 to 9 fluorine, chlorine and/or bromine atoms; methoxycarbonyl, ethoxycarbonyl, phenyl, —(CH₂)ₘ—SO₂R¹¹ or —SO₂NR¹²R¹³,
   or either R⁷ and R⁸ or R⁸ and R⁹ or R⁹ and R¹⁰ together form a further saturated or unsaturated 5- or 6-membered ring which again may contain one or two heteroatom groupings from the group consisting of N, O, S and SO₂,
m represents 0 or 1,
R¹¹ represents methyl, ethyl, n-propyl, i-propyl, n-butyl, i-butyl, s-butyl, t-butyl or morpholino,
R¹² represents methyl, ethyl, n-propyl, i-propyl, n-butyl, i-butyl, s-butyl, t-butyl or represents phenyl which is optionally mono- to trisubstituted by identical or different substituents from the group consisting of fluorine, chlorine, bromine, methyl, ethyl, n-propyl, i-propyl, n-butyl, i-butyl, s-butyl, t-butyl, methoxy, ethoxy, n-propoxy, i-propoxy, n-butoxy, i-butoxy, s-butoxy, t-butoxy,
R¹³ represents hydrogen or methyl, ethyl, n-propyl, i-propyl, n-butyl, i-butyl, s-butyl, t-butyl.

Very particular preference is given to Δ¹-pyrrolines of the formula (I) in which
R¹ represents fluorine, chlorine or methyl,
R² represents hydrogen, fluorine or chlorine,
R³ represents hydrogen, fluorine, chlorine, methyl, methoxy or ethoxy,
R⁴, R⁵ and R⁶ each represent hydrogen,
X represents O (oxygen),
Y represents CR¹⁰ or N (nitrogen),
R⁷, R⁸, R⁹ and R¹⁰ independently of one another represent hydrogen, fluorine, chlorine, bromine, nitro, methyl, i-propyl, t-butyl, trifluoromethyl, trifluoromethoxy, trifluoromethylthio, methoxycarbonyl, phenyl, —SO₂-morpholino, —CH₂SO₂Me, —SO₂NHMe, —SO₂NMe₂, —SO₂NH-(3,4-dichlorophenyl) or —SO₂NH-(2-methoxyphenyl).

Preference is furthermore given to Δ¹-pyrrolines of the formula (I) in which R¹ and R² represent fluorine.

Preference is furthermore given to Δ¹-pyrrolines of the formula (I) in which Y represents CR¹⁰ and particularly preferably represents CH.

Preference is furthermore given to Δ¹-pyrrolines of the formula (I) in which R³ represents hydrogen or fluorine.

Preference is furthermore given to Δ¹-pyrrolines of the formula (I) in which X represents O (oxygen).

Preference is furthermore given to Δ¹-pyrrolines of the formula (I) in which X represents S (sulphur).

Preference is furthermore given to Δ¹-pyrrolines of the formula (I) in which R⁴ and R⁵ represent hydrogen.

Preference is furthermore given to Δ¹-pyrrolines of the formula (I) in which R¹ and R² represent fluorine, X represents O (oxygen) and Y represents CH.

Very particular preference is given to (R)-configured compounds of the formula (I-a) 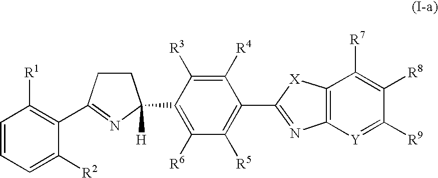
in which
R¹, R², R³, R⁴, R⁵, R⁶, X, Y, R⁷, R⁸ and R⁹ are as defined above.

Compounds of the formula (I-a) are obtained by customary processes for optical resolution, such as, for example, by chromatography of the corresponding racemates on a chiral stationary phase. In this manner, it is possible to resolve both racemic end products and racemic intermediates into the two enantiomers.

As far as this is possible, saturated hydrocarbon radicals, such as alkyl, can in each case be straight-chain or branched.

However, the general or preferred radical definitions or illustrations listed above can also be combined with one another, i.e. between the respective ranges and preferred ranges. The definitions apply both to the end products and, correspondingly to precursors and intermediates.

Using 4-[5-(2,6-difluorophenyl)-3,4-dihydro-2H-pyrrol-2-yl]-N-(2-hydroxy-phenyl)benzamide as starting material in the presence of p-toluenesulphonic acid (TsOH), the course of the process (A) according to the invention can be illustrated by the formula scheme below. 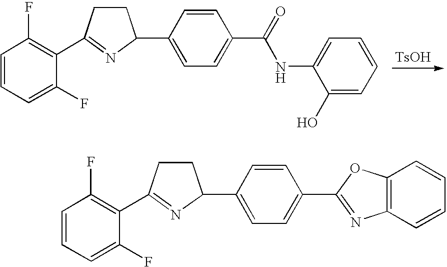
Explanation of the Processes and Intermediates
Process (A)

The formula (II) provides a general definition of the starting materials required for carrying out the process (A) according to the invention. In this formula, R¹, R², R³, R⁴, R⁵, R⁶, X, Y, R⁷, R⁸ and R⁹ preferably, particularly preferably and very particularly preferably have those meanings which have already been mentioned in connection with the description of the compounds of the formula (I) according to the invention as being preferred, particularly preferred, etc., for these radicals.

The compounds of the formula (II) are novel. They also have insecticidal action (cf. the Use Examples).

Compounds of the formula (II) can be prepared by
a) reacting Δ¹-pyrrolines of the formula (III) 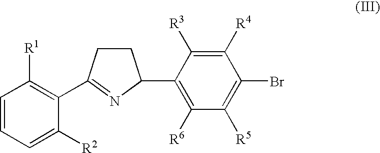
   in which
R¹, R², R³, R⁴, R⁵ and R⁶ are as defined above,
with an aniline derivative of the formula (IV) 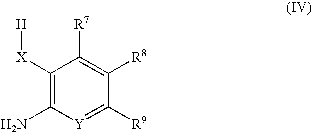
   in which
X, Y, R⁷, R⁸ and R⁹ are as defined above,
in the presence of carbon monoxide, if appropriate in the presence of a diluent j (for example toluene or dimethylformamide), if appropriate in the presence of an acid binder (for example 1,8-diazabicyclo[5.4.0]undec-7-ene, DBU) and, if appropriate, in the presence of a catalyst (for example PdCl₂/PPh₃/dppp [dppp=1,3-bis(diphenylphosphino)propane]), or
b) reacting Δ¹-pyrrolines of the formula (V) 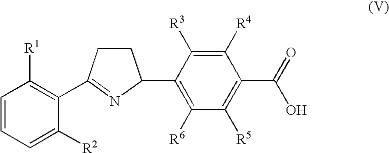
   in which
R¹, R², R³, R⁴, R⁵ and R⁶ are as defined above,
with an aniline derivative of the formula (IV) 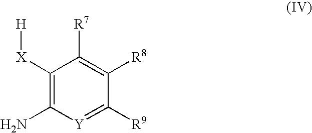
   in which
X, Y, R⁷, R⁸ and R⁹ are as defined above,
if appropriate in the presence of a base (for example diisopropylethylamine=Hünig's base) and, if appropriate, in the presence of a reaction auxiliary (for example bis-(2-oxo-oxazolidinyl)phosphoryl chloride=BOP—Cl) and, if appropriate, in the presence of a diluent.

The formula (III) provides a general definition of the Δ¹-pyrrolines required as starting materials, for carrying out the process (a) according to the invention. In this formula, R¹, R², R³, R⁴, R⁵ and R⁶ preferably, particularly preferably and very particularly preferably have those meanings which have already been mentioned in connection with the description of the compounds of the formula (I) according to the invention as being preferred, particularly preferred, etc., for these radicals.

Δ¹-Pyrrolines of the formula (V) are known and/or can be prepared by known processes (cf. WO 98/22438 and DE10047109.9).

The formula (V) provides a general definition of the Δ¹-pyrrolines required as starting materials for carrying out the process (b) according to the invention. In this formula, R¹, R², R³, R⁴, R⁵ and R⁶ preferably, particularly preferably and very particularly preferably have those meanings which have already been mentioned in connection with the description of the compounds of the formula (I) according to the invention as being preferred, particularly preferred, etc., for these radicals.

Δ¹-Pyrrolines of the formula (V) are known and/or can be prepared by known processes (cf. WO 98/22438 and DE10047109.9).

Δ¹-Pyrrolines of the formula (V) can also be prepared by
c) reacting Δ¹-pyrrolines of the formula (III) 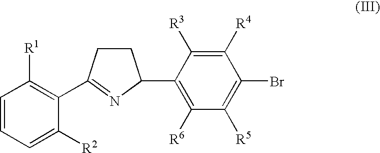
   in which
R¹, R², R³, R⁴, R⁵ and R⁶ are as defined above,
in a first step with an alcohol of the formula (VI)
   R¹⁴—OH (VI)
   in which
R¹⁴ represents C₂-C₆-alkyl, preferably C₂-C₄-alkyl, particularly preferably ethyl or n-butyl, very particularly preferably n-butyl,
in the presence of carbon monoxide, in the presence of a base (for example diisopropylethylamine) and in the presence of a catalyst (for example PdCl₂/PPh₃/dppp)
and, in a second step, hydrolyzing the resulting Δ¹-pyrrolines of the formula (VII) 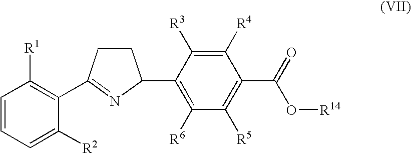
   in which
R¹⁴ is as defined above
in the presence of an acid (for example 20% strength HCl) and, if appropriate,
in the presence of a diluent (for example ethylene glycol dimethyl ether).

The formula (IV) provides a general definition of the aniline derivatives required as starting materials for carrying out the processes (a) and (b) according to the invention. In this formula, X, Y¹, R⁷, R⁸ and R⁹ preferably, particularly preferably and very particularly preferably have those meanings which have already been mentioned in connection with the description of the compounds of the formula (I) according to the invention as being preferred, particularly preferred, etc., for these radicals.

Aniline derivatives of the formula (IV) are known and/or can be prepared by known processes.

Aniline derivatives of the formula (IV) can be prepared, for example, from the analogous nitroaryl compounds (cf. WO 99/32436). The reduction of the nitro group to the amine is carried out either by using a metal catalyst (for example Ni, Pd, Pt) in the presence of hydrogen or a hydride donor (for example formate, cyclohexadiene, borohydride) (cf. Rylander: Hydrogenation Methods; Academic Press, London 1985). Nitroaryls can also be reduced directly by employing a strong hydride source (for example LiAnH₄, cf. Seyden-Penne: Reductions by the Alumino- and Borohydrides in Organic Synthesis; VCH Publishers, New York 1991) or by using a zerovalent metal (for example Fe, Sn, Ca), if appropriate in the presence of an acid. The nitroaryl compounds are obtained by known processes (cf. March: Advanced Organic Chemistry, 3^{rd} Ed., John Wiley, New York 1985).

The Δ¹-pyrrolines of the formula (III) required as starting materials for carrying out the process (c) according to the invention have already been described above.

The alcohols of the formula (VI) required as starting materials for carrying out the process (c) according to the invention are known.

Suitable diluents for carrying out the process (A) according to the invention are all customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichloroethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; nitriles, such as acetonitrile, propionitrile, n- or isobutyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; or sulphones, such as sulpholane. Particular preference is given to using acetone, dimethoxyethane, dioxane, tetrahydrofuran, dimethylformamide, dimethylacetamide, dimethyl sulphoxide, ethanol, toluene or, if appropriate, mixtures of the diluents mentioned with water. Particular preference is given to using toluene or benzene.

When carrying out the process (A) according to the invention, the reaction temperatures can be varied within a relatively wide range. In general, the process is carried out at temperatures between 0° C. and 140° C., preferably between 20° C. and 120° C., particularly preferably between 80° C. and 120° C.

All of the processes according to the invention are generally carried out under atmospheric pressure. However, in each case it is also possible to operate under elevated or reduced pressure.

When carrying out the process (A) according to the invention, in general from 2 to 4 mol of p-toluenesulphonic acid are employed per mole of the compound of the formula (II). However, it is also possible to employ the reaction components in other ratios. Work-up is carried out by customary methods. In general, the reaction mixture is diluted and extracted with an organic solvent. The organic phase is washed, dried, filtered and concentrated. If appropriate, the residue is freed of any impurities that may still be present using customary methods, such as chromatography or recrystallization.

Chiral Compounds of the Formula (I-a)

To produce chiral compounds of the formula (I-a), it is possible, for example, to subject the Δ¹-pyrrolines, used as intermediates, of the formula (III) 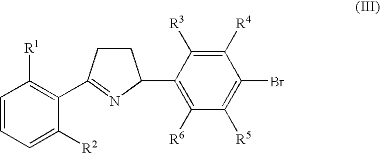
in which
R¹, R², R³, R⁴, R⁵ and R⁶ are as defined above,
to an optical resolution. This is effected, for example, using methods of preparative chromatography, preferably the High Performance Liquid Chromatography (HPLC) method. For this purpose, a chiral stationary silica gel phase is used. A tris-(3,5-dimethylphenylcarbamate) cellulose-modified silica gel has been found to be particularly suitable for separating the compounds of the formula (III) into the two enantiomers. This separation material is commercially available. However, it is also possible to use other stationary phases. Suitable eluents are all customary inert organic solvents, and mixtures of these. Preference is given to using optionally halogenated aliphatic, alicyclic or aromatic hydrocarbons, such as petroleum ether, hexane, heptane, cyclohexane; dichloromethane, chloroform; alcohols, such as methanol, ethanol, propanol; nitriles, such as acetonitrile; esters, such as methyl acetate or ethyl acetate. Particular preference is given to using aliphatic hydrocarbons, such as hexane or heptane, and alcohols, such as methanol or propanol, very particularly preferably n-heptane and isopropanol, or mixtures of these. In general, this is effected at temperatures between 10° C. and 60° C., preferably between 10° C. and 40° C., particularly preferably at room temperature. The (R)-configured enantiomers obtained in this manner are then used as starting materials for process (a) or (c).

The active compounds, having good plant tolerance, favourable homeotherm toxicity and good environmental compatibility, are suitable for protecting plants and plant organs, for increasing harvest yields, for improving the quality of the harvested produce and for controlling animal pests, in particular insects, arachnids and nematodes, which are encountered in agriculture, in forests, in gardens and leisure facilities, in the protection of stored products and of materials, and in the hygiene sector. They are preferably used as crop protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example; Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae and Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothrips femoralis, Thrips tabaci, Thrips palmi and Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp. and Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

The compounds of the formula (I) according to the invention are particularly effective against caterpillars, beetle larvae, spider mites, aphids and leaf-mining flies.

The compounds according to the invention additionally also have very good persistency, such as, for example, against the caterpillars of the cotton budworm (Heliothis virescens) or the caterpillars of the armyworm (Spodoptera frugiperda).

At certain concentrations or application rates, the compounds according to the invention may, if appropriate, also be used as herbicides and microbicides, for example as fungicides, antimycotics and bactericides. If appropriate, they may also be used as intermediates or precursors for the synthesis of further active compounds.

All plants and plant parts can be treated in accordance with the invention. Plants are to be understood as meaning in the present context all plants and plant populations such as desired and undesired wild plants or crop plants (inclusive of naturally occurring crop plants). Crop plants can be plants which can be obtained by conventional plant breeding and optimization methods or by biotechnological and recombinant methods or by combinations of these methods, inclusive of the transgenic plants and inclusive of the plant cultivars protectable or not protectable by plant breeders' rights. Plant parts are to be understood to mean all above-ground and underground parts and organs of plants, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted into the customary formulations, such as solutions, emulsions, wettable powders, suspensions, powders, dusts, pastes, soluble powders, granules, suspension-emulsion concentrates, natural and synthetic materials impregnated with active compound, and microencapsulations in polymeric substances.

These formulations are produced in a known manner, for example by mixing the active compounds with extenders, that is liquid solvents and/or solid carriers, optionally with the use of surfactants, that is emulsifiers and/or dispersants, and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

Suitable solid carriers are:
for example ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks such as calcite, marble, pumice, sepiolite and dolomite, and also synthetic granules of inorganic and organic mieals, and granules of organic material such as sawdust, coconut shells, maize cobs and tobacco stalks; suitable emulsifiers and/or foam-formers are: for example nonionic and anionic emulsifiers, such as polyoxyethylene fatty acid esters, polyoxyethylene, fatty alcohol ethers, for example alkylaryl polyglycol ethers, alkylsulphonates, alkyl sulphates, arylsulphonates and also protein hydrolysates; suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other additives can be mineral and vegetable oils.

It is possible to use colorants such as inorganic pigments, for example iron oxide, titanium oxide and Prussian Blue, and organic dyes, such as alizarin dyes, azo dyes and metal phthalocyanine dyes, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention can be used as such or in their formulations as a mixture with known fungicides, bactericides, acaricides, nematicides or insecticides in order to extend the spectrum of activity or prevent resistances developing. In many cases, synergistic effects are thus obtained, ie. the efficacy of the mixture is greater than the efficacy of the individual components.

Examples of Co-Components in Mixtures are the Following Compounds:

Fungicides:

aldimorph, ampropylfos, ampropylfos-potassium, andoprim, anilazine, azaconazole, azoxystrobin,

benalaxyl, benodanil, benomyl, benzamacril, benzamacril-isobutyl, bialaphos, binapacryl, biphenyl, bitertanol, blasticidin-S, bromuconazole, bupirimate, buthiobate,

calcium polysulphide, carpropamide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,

debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

edifenphos, epoxiconazole, etaconazole, ethirimol, etridiazole,

famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox,

guazatine,

hexachlorobenzene, hexaconazole, hymexazole,

imazalil, imibenconazole, iminoctadine, iminoctadine albesilate, iminoctadine triacetate, iodocarb, ipconazole, iprobenfos (IBP), iprodione, iprovalicarb, irumamycin, isoprothiolane, isovaledione,

kasugamycin, kresoxim-methyl, copper preparations, such as: copper hydroxide, copper naphthenate, copper oxychloride, copper sulphate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur,

quinconazole, quintozene (PCNB), quinoxyfen,

sulphur and sulphur preparations, spiroxamines,

tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole, uniconazole,

validamycin A, vinclozolin, viniconazole,

zarilamide, zineb, ziram and also

Dagger G,

OK-8705, OK-8801,

α-(1,1-dimethylethyl)-β-(2-phenoxyethyl)-1H-1,2,4-triazole-1-ethanol,

α-(2,4-dichlorophenyl)-β-fluoro-β-propyl-1H-1,2,4-triazole-1-ethanol,

α-(2,4-dichlorophenyl)-β-methoxy-α-methyl-1H-1,2,4-triazole-1-ethanol,

α-(5-methyl-1,3-dioxan-5-yl)-β-[[4-(trifluoromethyl)-phenyl]-methylene]-1H-1,2,4-triazole-1-ethanol,

(5RS,6RS)-6-hydroxy-2,2,7,7-tetramethyl-5-(1H-1,2,4-triazol-1-yl)-3-octanone,

(E)-α-(methoxyimino)-N-methyl-2-phenoxy-phenylacetamide,

1-(2,4-dichlorophenyl)-2-(1H-1,2,4-triazol-1-yl)-ethanone-O-(phenylmethyl)-oxime,

1-(2-methyl-1-naphthalenyl)-1-pyrrole-2,5-dione,

1-(3,5-dichlorophenyl)-3-(2-propenyl)-2,5-pyrrolidinedione,

1-[(diiodomethyl)-sulphonyl]-4-methyl-benzene,

1-[[2-(2,4-dichlorophenyl)-1,3-dioxolan-2-yl]-methyl]-1H-imidazole,

1-[[2-(4-chlorophenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinol,

2′,6′-dibromo-2-methyl-4′-trifluoromethoxy-4′-trifluoro-methyl-1,3-thiazole-5-carboxanilide,

2,6-dichloro-5-(methylthio)-4-pyrimidinyl-thiocyanate,

2,6-dichloro-N-(4-trifluoromethylbenzyl)-benzamide,

2,6-dichloro-N-[[4-(trifluoromethyl)-phenyl]-methyl]-benzamide,

2-(2,3,3-triiodo-2-propenyl)-2H-tetrazole,

2-[(1-methylethyl)-sulphonyl]-5-(trichloromethyl)-1,3,4-thiadiazole,

2-[[6-deoxy-4-O-(4-O-methyl-β-D-glycopyranosyl)-α-D-glucopyranosyl]-amino]-4-methoxy-1H-pyrrolo[2,3-d]pyrimidine-5-carbonitrile,

2-aminobutane,

2-bromo-2-(bromomethyl)-pentanedinitrile,

2-chloro-N-(2,3-dihydro-1,1,3-trimethyl-1H-inden-4-yl)-3-pyridinecarboxamide,

2-chloro-N-(2,6-dimethylphenyl)-N-(isothiocyanatomethyl)-acetamide,

2-phenylphenol (OPP),

3,4-dichloro-1-[4-(difluoromethoxy)-phenyl]-1H-pyrrole-2,5-dione,

3,5-dichloro-N-[cyano-[(1-methyl-2-propynyl)-oxy]-methyl]-benzamide,

3-(1,1-dimethylpropyl)-1-oxo-1H-indene-2-carbonitrile,

3-[2-(4-chlorophenyl)-5-ethoxy-3-isoxazolidinyl]-pyridine,

4-chloro-2-cyano-N,N-dimethyl-5-(4-methylphenyl)-1H-imidazole-1-sulphonamide,

4-methyl-tetrazolo[1,5-α]quinazolin-5(4H)-one,

8-hydroxyquinoline sulphate,

9H-xanthene-2-[(phenylamino)-carbonyl]-9-carboxylic hydrazide,

bis-(1-methylethyl)-3-methyl4-[(3-methylbenzoyl)-oxy]-2,5-thiophenedicarboxylate,

cis-1-(4-chlorophenyl)-2-(1H-1,2,4-triazol-1-yl)-cycloheptanol,

cis-4-[3-[4-(1,1-dimethylpropyl)-phenyl-2-methylpropyl]-2,6-dimethyl-morpholinehydrochloride,

ethyl [(4-chlorophenyl)-azo]-cyanoacetate,

potassium hydrogen carbonate,

methanetetrathiol sodium salt,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,6-dimethylphenyl)-2-methoxy-N-(tetrahydro-2-oxo-3-furanyl)-acetamide,

N-(2,6-dimethylphenyl)-2-methoxy-N-(tetrahydro-2-oxo-3-thienyl)-acetamide,

N-(2-chloro-4-nitrophenyl)-4-methyl-3-nitro-benzenesulphonamide,

N-(4-cyclohexylphenyl)-1,4,5,6-tetrahydro-2-pyrimidinamine,

N-(4-hexylphenyl)-1,4,5,6-tetrahydro-2-pyrimidinamine,

N-(5-chloro-2-methylphenyl)-2-methoxy-N-(2-oxo-3-oxazolidinyl)-acetamide,

N-(6-methoxy)-3-pyridinyl)-cyclopropanecarboxamide,

N-[2,2,2-trichloro-1-[(chloroacetyl)-amino]-ethyl]-benzamide,

N-[3-chloro4,5-bis-(2-propinyloxy)-phenyl]-N′-methoxy-methanimidamide,

N-formyl-N-hydroxy-DL-alanine sodium salt,

O,O-diethyl [2-(dipropylamino)-2-oxoethyl]-ethylphosphoramidothioate,

O-methyl S-phenyl phenylpropylphosphoramidothioate,

S-methyl 1,2,3-benzothiadiazole-7-carbothioate,

spiro[2H]-1-benzopyrane-2,1′(3′H)-isobenzofuran-3′-one,

4-[(3,4-dimethoxyphenyl)-3-(4-fluorophenyl)acryloyl]morpholine.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:

abamectin, acephate, acetamiiprid, acequinocyl, acrinathrin, alanycarb, aldicarb, aldoxycarb, alpha-cypermethrin,. alphamethrin, amitraz, avermectin, AZ 60541, azadirachtin, azamethiphos, azinphos A, azinphos M, azocyclotin,

Bacillus popilliae, Bacillus sfphaericus, Bacillus subtilis, Bacillus thuringiensis, Baculoviruses, Beauveria bassiana, Beauveria tenella, bendiocarb, benfuracarb, bensultap, benzoximate, betacyfluthrin, bifenazate, bifenthrin, bioethanomethrin, bio-permethrin, bistrifluron, BPMC, bromophos A, bufencarb, buprofezin, butathiofos, butocarboxim, butylpyridaben,

cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine,

deltamethrin, demeton M, demeton S, demeton-S-methyl, diafenthiuron, diazinon, dichlorvos, dicofol, diflubenzuron, dimethoate, dimethylvinphos, dimethylvinphos, dinetofuran, diofenolan, disulfoton, docusat-sodium, dofenapyn,

eflusilanate, emamectin, empenthrin, endosulfan, Entomopfthora spp., esfenvalerate, ethiofencarb, ethion, ethiprole, ethoprophos, etofenprox, etoxazole, etrimfos,

fenamiphos, fenazaquin, fenbutatin oxide, fenitrothion, fenothiocarb, fenoxacrim, fenoxycarb, fenpropathrin, fenpyrad, fenpyrithrin, fenpyroximate, fenthion, fenvalerate, fipronil, fluazuron, flubrocythrinate, flucycloxuron, flucythrinate, flufenoxuron, flumethrin, flupyrazofos, flutenzine, fluvalinate, fonophos, fosmethilan, fosthiazate, fubfenprox, furathiocarb,

granulosis viruses,

halofenozide, HCH, heptenophos, hexaflumuron, hexythiazox, hydroprene, imidacloprid, indoxacarb, isazofos, isofenphos, isoxathion, ivermectin, nuclear polyhedrosis viruses,

lambda-cyhalothrin, lufenuron,

malathion, mecarbam, metaldehyde, methamidophos, metharhizium anisopliae, metharhizium flavoviride, methidathion, methiocarb, methoprene, methomyl, methoxyfenozide, metolcarb, metoxadiazone, mevinphos, milbemectin, milbemycin, monocrotophos,

naled, nitenpyram, nithiazine, novaluron,

omethoate, oxamyl, oxydemethon M,

Paecilomyces fumosoroseus, parathion A, parathion M, permethrin, phenthoate, phorate, phosalone, phosmet, phosphamidon, phoxim, pirimicarb, pirimiphos A, pirimiphos M, profenofos, promecarb, propargite, propoxur, prothiofos, prothoate, pymetrozine, pyraclofos, pyresmethrin, pyrethrum, pyridaben, pyridathion, pyrimidifen, pyriproxyfen,

quinalphos,

ribavirin,

salithion, sebufos, silafluofen, spinosad, spirodiclofen, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, tetradifon, thetacypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazurone, trichlophenidine, trichlorfon, triflumuron, trimethacarb,

vamidothion, vaniliprole, Verticillium lecanii,

YI 5302,

zeta-cypermethrin, zolaprofos,

(1R-cis)-[5-(phenylmethyl)-3-furanyl]-methyl 3-[(dihydro-2-oxo-3(2H)-furanylidene)-methyl]-2,2-dimethylcyclopropanecarboxylate,

(3-phenoxyphenyl)-methyl 2,2,3,3-tetramethylcyclopropanecarboxylate,

1-[(2-chloro-5-thiazolyl)methyl]tetrahydro-3,5-dimethyl-N-nitro-1,3,5-triazine-2(1H)-imine,

2-(2-chloro-6-fluorophenyl)4-[4-(1,1-dimethylethyl)phenyl]4,5-dihydro-oxazole, 2-(acetyloxy)-3-dodecyl-1,4-naphthalenedione,

2-chloro-N-[[[4-(1-phenylethoxy)-phenyl]-amino]-carbonyl]-benzamide,

2-chloro-N-[[[4-(2,2-dichloro-1,1-difluoroethoxy)-phenyl]-amino]-carbonyl]-benzamide,

3-methylphenyl propylcarbamate,

4-[4-(4-ethoxyphenyl)-4-methylpentyl]-1-fluoro-2-phenoxy-benzene,

4-chloro-2-(1,1-dimethylethyl)-5-[[2-(2,6-dimethyl-4-phenoxyphenoxy)ethyl]thio]-3(2H)-pyridazinone,

4-chloro-2-(2-chloro-2-methylpropyl)-5-[(6-iodo-3-pyridinyl)methoxyl-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

[2-benzoyl-1-(1,1-dimethylethyl)-hydrazinobenzoic acid,

2,2-dimethyl-3-(2,4-dichlorophenyl)-2-oxo-1-oxaspiro[4.5]dec-3-en-4-yl butanoate,

[3-[(6-chloro-3-pyridinyl)methyl]-2-thiazolidinylidene]-cyanamide,

dihydro-2-(nitromethylene)-2H-1,3-thiazine-3(4H)-carboxaldehyde,

ethyl [2-[[1,6-dihydro-6-oxo-1-(phenylmethyl)-4-pyridazinyl]oxy]ethyl]-carbamate,

N-(3,4,4-trifluoro-1-oxo-3-butenyl)-glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitro-guanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl [2-(dipropylamino)-2-oxoethyl]-ethylphosphoramidothioate.

N-cyanomethyl4-trifluoromethylnicotinamide,

3,5-dichloro-1-(3,3-dichloro-2-propenyloxy)4-[3-(5-trifluoromethylpyridin-2-yloxy)propoxy]benzene.

A mixture with other known active compounds, such as herbicides, or with fertilizers and growth regulators, safeners or semiochemicals is also possible.

The active compounds according to the invention, can furthermore be present when used as insecticides in their commercially available formulations and in the use forms, prepared from these formulations, as a mixture with synergistic agents. Synergistic agents are compounds which increase the effect of the active compounds, without the added synergistic agent itself having to be actively effective.

The active compounds according to the invention can furthermore be present when used as insecticides in their commercially available formulations and in the use forms, prepared from these formulations, as a mixture with inhibitors which reduce degradation of the active compound after use in the vicinity of the plant, on the surface of parts of plants or in plant tissues.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as a good stability to alkali on limed substrates.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant part's” has been explained above.

Particularly preferably, plants of the plant cultivars which are in each case commercially available or ink use are treated according to the invention. Plant cultivars are to be understood as meaning plants, having novel properties (“traits”) which have been obtained by conventional breeding, by mutagenesis or by recombinant DNA techniques. This can be varieties, bio- and genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars to be treated (i.e. those obtained by genetic engineering) which are preferred according to the invention include all plants which, in the genetic modification, received genetic material which imparted particularly advantageous useful traits to these plants. Examples of such traits are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, tobacco, oilseed rape and also fruit plants (with the fruits apples, pears, citrus fruits and grapevines), and particular emphasis is given to maize, soya beans, potatoes, cotton, tobacco and oilseed rape. Traits that are emphasized are in particular increased defence of the plants against insects, arachnids, nematodes and snails by toxins formed in the plants, in particular those formed by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) in the plants (hereinbelow referred to as “Bt plants”). Traits that are also particularly emphasized are the increased defence of the plants to fungi, bacteria and viruses by systemic acquired resistance (SAR), systemin, phytoalexius, elicitous and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Boligard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these or still-to-be-developed genetic traits, which plants will be developed and/of marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or the mixtures specifically mentioned in the present text.

The active compounds according to the invention act not only against plant, hygiene and stored product pests, but also in the veterinary medicine sector against animal parasites (ectoparasites), such as hard ticks, soft ticks, mange mites, leaf mites, flies (biting and licking), parasitic fly larvae, lice,. hair lice, feather lice and fleas. These parasites include:

From the order of the Anoplurida, for example, Haematopinus spp., Linognathus spp., Pediculus spp., Phtirus spp. and Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.

From the order Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopsylla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattela germanica and Supella spp.

From the subclass of the Acaria (Acarida) and the orders of the Meta- and Mesostigmata, for example, Argas spp., Omithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injections (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) according to the invention can be used as formulations (for example powders, emulsions, free-flowing compositions), which comprise the active compounds according to the invention in an amount of 1 to 80% by weight, directly or after 100 to 10 000-fold dilution, or they can be used as a chemical bath.

It has furthermore been found that the compounds according to the invention also have a strong insecticidal action against insects which destroy industrial materials.

The following insects may be mentioned as examples and as being preferred—but without any limitation:

Beetles, such as

Hylotrupes bajulus, Chlorophorus pilosis, Anobium punctatum, Xestobium rufovillosum, Ptilinus pecticornis, Dendrobium pertinex, Emobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec. and Dinoderus minutus.

Hymenopterons, such as

Sirex juvencus, Urocerus gigas, Urocerus gigas taignus and Urocerus augur.

Termites, such as

Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus.

Bristletails, such as Lepisma saccharina.

Industrial materials in the present context are to be understood as meaning non-living materials, such as, preferably, plastics, adhesives, sizes, papers and cards, leather, wood and processed wood products and coating compositions.

Wood and processed wood products are materials to be protected, especially preferably, from insect infestation.

Wood and processed wood products which can be protected by the agent according to the invention or mixtures comprising this are to be understood as meaning, for example:
building timber, wooden beams, railway sleepers, bridge components, boat jetties, wooden vehicles, boxes, pallets, containers, telegraph poles, wood panelling, wooden windows and doors, plywood, chipboard, joinery or wooden products which are used quite generally in house-building or in building joinery.

The active compounds can be used as such, in the form of concentrates or in generally customary formulations, such as powders, granules, solutions, suspensions, emulsions or pastes.

The formulations mentioned can be prepared in a manner known per se, for example by mixing the active compounds with at least one solvent or diluent, emulsifier, dispersing agent and/or binder or fixing agent, a water repellent, if appropriate siccatives and UV stabilizers and if appropriate dyes and pigments, and also other processing auxiliaries.

The insecticidal compositions or concentrates used for the preservation of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by a series of tests. In general, however, it is sufficient to employ 0.0001 to 20% by weight, preferably 0.001 to 10% by weight, of the active compound, based on the material to be preserved.

Solvents and/or diluents which are used are an organic chemical solvent or solvent mixture and/or an oily or oil-like organic chemical solvent or solvent mixture of low volatility and/or a polar organic chemical solvent or solvent mixture and/or water, and if appropriate an emulsifier and/or wetting agent.

Organic chemical solvents which are preferably used are oily or oil-like solvents having an evaporation number above 35 and a flashpoint above 30° C., preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkylbenzene.

Mineral oils having a boiling range from 170 to 220° C., white spirit having a boiling range from 170 to 220° C., spindle oil having a boiling range from 250 to 350° C., petroleum and aromatics having a boiling range from 160 to 280° C., turpentine oil and the like, are advantageously employed.

In a preferred embodiment, liquid aliphatic hydrocarbons having a boiling range from 180 to 210° C. or high-boiling mixtures of aromatic and aliphatic hydrocarbons having a boiling range from 180 to 220° C. and/or spindle oil and/or monochloronaphthalene, preferably α-monochloronaphthalene, are used.

The organic oily or oil-like solvents of low volatility which have an evaporation number above 35 and a flashpoint above 30° C., preferably above 45° C., can be replaced in part by organic chemical solvents of high or medium volatility, providing that the solvent mixture likewise has an evaporation number above 35 and a flashpoint above 30° C., preferably above 45° C., and that the insecticide/fungicide mixture is soluble or emulsifiable in this solvent mixture.

According to a preferred embodiment, some of the organic chemical solvent or solvent mixture is replaced by an aliphatic polar organic chemical solvent or solvent mixture. Aliphatic organic chemical solvents containing hydroxyl and/or ester and/or ether groups, such as, for example, glycol ethers, esters or the like, are preferably used.

Organic chemical binders which are used in the context of the present invention are the synthetic resins and/or binding drying oils which are known per se, are water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-cumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odour correctants and inhibitors or anticorrosive agents and the like which are known per se can additionally be employed.

It is preferred according to the invention for the composition or concentrate to comprise, as the organic chemical binder, at least one alkyd resin or modified alkyd resin and/or one drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phthalic acid esters, such as dibutyl, dioctyl or benzyl butyl phthalate, phosphoric acid esters, such as tributyl phosphate, adipic acid esters, such as di-(2-ethylhexyl)adipate, stearates, such as butyl stearate or amyl stearate, oleates, such as butyl oleate, glycerol ethers or higher molecular weight glycol ethers, glycerol esters and p-toluenesulphonic acid esters.

Fixing agents are based chemically on polyvinyl alkyl ethers, such as, for example, polyvinyl methyl ether or ketones, such as benzophenone or ethylenebenzophenone. Possible solvents or diluents are, in particular, also water, if appropriate as a mixture with one or more of the abovementioned organic chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Especially preferred mixing partners which may be mentioned are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyphenoxide, triflumuron, clothianidin, spinosad, tefluthrin and triflumuron,
and also fungicides, such as epoxyconazole, hexaconazole, azaconazole, propiconazole, tebuconazole, cyproconazole, metconazole, imazalil, dichlorfluanid, tolylfluanid, 3-iodo-2-propinyl-butyl carbamate, N-octyl-isothiazolin-3-one and 4,5-dichloro-N-octylisothiazolin-3-one.

The compounds according to the invention can at the same time be employed for protecting objects which come into contact with salt water or brackish water, such as hulls, screens, nets, buildings, moorings and signalling systems, against fouling.

Fouling by sessile Oligochaeta, such as Serpulidae, and by shells and species from the ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Cerarnium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis-(trialkyltin)sulphides, tri-n-butyltin laurate, tri-n-butyltin chloride, copper(I)oxide, triethyltin chloride, tri-n-butyl-(2-phenyl-4-chlorophenoxy)tin, tributyltin oxide, molybdenum disulphide, antimony oxide, polymeric butyl titanate, phenyl-(bispyridine)-bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I)ethylene-bisdithiocarbamate, copper thiocyanate, copper naphthenate and tributyltin halides to be dispensed with, or the concentration of these compounds to be substantially reduced.

If appropriate, the reedy-to-use antifouling paints can additionally comprise other active compounds, preferably algicides, fungicides, herbicides, molluscicides, or other antifouling active compounds.

Preferably suitable components in combinations with the antifouling compositions according to the invention are:
algicides such as
2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;
fungicides such as
benzo[b]thiophenecarboxylic acid cyclohexylamide S,S-dioxide, dichlofluanid, fluor-folpet, 3-iodo-2-propinyl butylcarbamate, tolylfluanid and azoles such as azaconazole, cyproconazole, epoxyconazole, hexaconazole, metconazole, propiconazole and tebuconazole;
molluscicides such as
fentin acetate, metaldehyde, methiocarb, niclosamid, thiodicarb and trimethacarb and Fe-chelate;
or conventional antifouling active compounds such as
4,5-dichloro-2-octyl-4-isothiazolin-3-one, diiodomethylparatryl sulphone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphonyl)-pyridine, 2,4,5,6-tetrachloroisophthalonitrile, tetra-methylthiuram disulphide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as colophonium to allow controlled release of the active compounds. Furthermore, the paints may comprise plasticizers, modifiers which affect the theological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Omithodorus moubat, Rhipicephalus sanguineus, Trombicula alfreddugesi, Neutrombicula autumnalis, Dermatophagoides pteronissimus and Dermatophagoides forinae.

From the order of the Araneae, for example, Aviculariidae and Araneidae.

From the order of the Opilionep, for example, Pseudoscorpiones chelifer, Pseudoscorpiones cheiridium and Opiliones phalangium.

From the order of the Isopoda, for example, Oniscus asellus and Porcellio scaber.

From the order of the Diplopoda, for example, Blaniulus guttulatus and Polydesmus spp.

From the order of the Chilopoda, for example, Geophilus spp.

From the order of the Zygentoma, for example, Ctenolepisma spp., Lepisma saccharina and lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa and Supella longipalpa.

From the order of the Saltatoria, for example, Acheta domesticus.

From the order of the Dermaptera, for example, Forficula auricularia.

From the order of the Isoptera, for example, Kalotermes spp. and Reticulitermes spp.

From the order of the Psocoptera, for example, Lepinatus spp. and Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais and Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga camaria, Simulium spp., Stomoxys calcitrans and Tipula paludosa.

From the order of the Lepidoptera, for example, Achroia grisella, Galleria mellonella, Plodia interpunctella, Tinea cloacella, Tinea pellionella and Tineola bisselliella.

From the order of the Siphonaptera, for example, Ctenocephalides canis, Ctenocephalides felis, Pulex irritans, Tunga penetrans and Xenopsylla cheopis.

From the order of the Hymenoptera, for example, Camponotus herculeanus, Lasius fuliginosus, Lasius niger, Lasius umbratus, Monomorium pharaonis, Paravespula spp. and Tetramorium caespitum.

From the order of the Anoplura, for example, Pediculus humanus capitis, Pediculus humanus corporis and Phthirus pubis.

From the order of the Heteroptera, for example, Cimex hemipterus, Cimex lectularius, Rhodinus prolixus and Triatoma infestans.

They are used in the household insecticides sector alone or in combination with other suitable active compounds such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The preparation and the use of the compounds according to the invention is shown in the examples below.

### PREPARATION EXAMPLES

### EXAMPLE 1

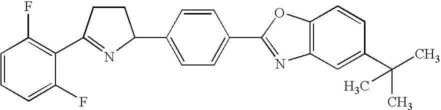

3.5 g of N-(5-tert-butyl-2-hydroxyphenyl)-4-[5-(2,6-difluorophenyl)-3,4-dihydro-2H-pyrrol-2-yl]benzamide (II-1) are dissolved in toluene, 4.13 g of p-toluenesulphonic acid are added and the mixture is, using a Soxleth with 3 Å molecular sieves, heated under reflux for 16 h. After cooling, the mixture is diluted with ethyl acetate and extracted with water/sodium bicarbonate solution. The organic phase is dried, filtered and concentrated. The crude product is purified by silica gel chromatography (mobile phase: ethyl acetate/cyclohexane 1:1).

This gives 0.9 g (26% of theory) of 5-tert-butyl-2-{4-[5-(2,6-difluorophenyl)-3,4-dihydro-2H-pyrrol-2-yl]phenyl}-1,3-benzoxazole.

HPLC: log P(2.3)=5.11

### EXAMPLE 2

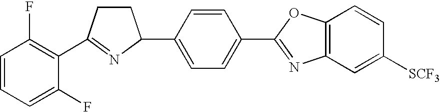

0.8 g of 4-[5-(2,6-difluorophenyl)-3,4-dihydro-2H-pyrrol-2-yl]-N-{2-hydroxy-5-[(tri-fluoromethyl)sulphanyl]phenyl}benzamide (II-2) is refluxed for 3 h with 0.8 g of p-toluenesulphonic acid in 120 ml of benzene using a Soxleth with 3 Å molecular sieves. After cooling, the mixture is concentrated, sodium bicarbonate is added to pH 8 and the mixture is stirred with aqueous sodium chloride solution and ethyl acetate. The mixture is extracted twice with ethyl acetate. The organic phase is dried, filtered and concentrated. The crude product is purified by silica gel chromatography (mobile phase: cyclohexane→cyclohexane/ethyl acetate 8:2).

This gives 0.37 g (48% of theory) of 2-{4-[5-(2,6-difluorophenyl)-3,4-dihydro-2H-pyrrol-2-yllphenyl}-5-[(trifluoromethyl)sulphanyl]-1,3-benzoxazole.

HPLC: log P (2.3)=4.98

### EXAMPLE 3

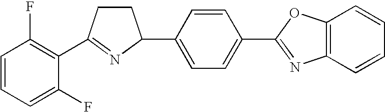

2 g of 4-[5-(2,6-difluorophenyl)-3,4-dihydro-2H-pyrrol-2-yl]-N-(2-hydroxyphenyl)-benzamide (II-3) and 3.5 g of p-toluenesulphonic acid in 120 ml of benzene are refluxed for 16 h using a Soxleth with 3 Å molecular sieves. After cooling, the mixture is concentrated, sodium bicarbonate is added to pH=8 and the mixture is stirred with aqueous sodium chloride solution and ethyl acetate. The mixture is extracted twice with ethyl acetate. The organic phase is dried, filtered and concentrated. The crude product is purified by silica gel chromatography (mobile phase: cyclohexane→cyclohexane/ethyl acetate 8:2).

This gives 1.14 g (59% of theory) of 2-{4-[5-(2,6-difluorophenyl)-3,4-dihydro-2H-pyrrol-2-yl]phenyl}-1,3-benzoxazole.

HPLC: log P (2.3)=3.22

¹H-NMR (CDCl₃): δ=8.25 (d, 2H), 7.75 (m, 1H), 7.55 (m, 1H), 7.5 (d, 2H), 7.35 (m, 3H), 7.0 (t, 2H), 5.55 (t, 1H), 3.1 (m, 2H), 2.75 (m, 1H), 1.9 (m, 1H) ppm.

### EXAMPLE 4

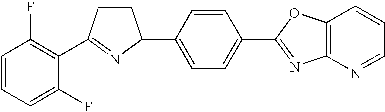

2 g of 4-[5-(2,6-difluorophenyl)-3,4-dihydro-2H-pyrrol-2-yl]-N-(3-hydroxy-2-pyridinyl)benzamide (II-4) and 2.93 g of p-toluenesulphonic acid in 100 ml of toluene are heated under reflux for 16 h using a Soxleth with 3 Å molecular sieves. The mixture is then neutralized by addition of sodium bicarbonate and the organic phase is dried over sodium sulphate, filtered and concentrated. This gives 0.86 g (44%) of 2-{4-[5-(2,6-difluorophenyl)-3,4-dihydro-2H-pyrrol-2-yl]-phenyl}[1,3]oxazole[4,5-b]pyridine.

HPLC: log P (2.3)=2.06

### EXAMPLE 5

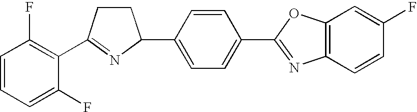

1.1 g of 4-[5-(2,6-difluorophenyl)-3,4-dihydro-2H-pyrrol-2-yl]-N-(5-fluor-2-hydroxy-phenyl)benzamide are dissolved in 100 ml of benzene and, together with 1.2 g of p-toluenesulphonic acid, heated under reflux using a Soxleth with 3 Å molecular sieves. After cooling, the mixture is diluted with ethyl acetate and neutralized with sodium bicarbonate. The organic phase is dried over sodium sulphate, filtered and concentrated. The crude product is purified by silica gel chromatography (mobile phase: ethyl acetate/cyclohexane 4:5).

This gives 0.24 g (30% of theory) of 2-{4-[5-(2,6-difluorophenyl)-3,4-dihydro-2H-pyrrol-2-yl]phenyl}-6-fluoro-1,3-benzoxazole.

HPLC: log P (2.3)=3.53

The following compounds of the formula (I) can be obtained analogously to Examples 1 to 5 and in accordance with the general description.

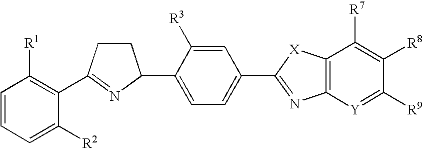

| No. | R¹ | R² | R³ | X | Y | R⁷ | R⁸ | R⁹ | R¹⁰ | log P |
|---|---|---|---|---|---|---|---|---|---|---|
| 6 | F | F | H | O | CR¹⁰ | H | NO₂ | Cl | H | 4.12^{a)} |
| 7 | F | F | H | O | CR¹⁰ | H | H | Cl | H | 4.23^{a)} |
| 8 | F | F | H | O | CR¹⁰ | H | Me | H | H | |
| 9 | F | F | H | O | CR¹⁰ | H | H | Me | H | |
| 10 | F | F | H | O | CR¹⁰ | H | 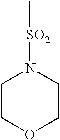 | H | H | |
| 11 | Me | H | H | O | CR¹⁰ | H | H | H | H | 2.25^{a)} |
| 12 | F | F | H | O | CR¹⁰ | Cl | H | Cl | H | 5.44^{a)} |
| 13 | F | F | H | O | CR¹⁰ | H | Cl | CF₃ | H | |
| 14 | F | F | F | O | CR¹⁰ | H | H | H | H | 4.25^{a)} |
| 15 | F | F | H | O | CR¹⁰ | H | SO₂NMe₂ | Cl | H | |
| 16 | F | F | H | O | CR¹⁰ | H | H | H | Me | 3.90^{a)} |
| 17 | F | F | H | O | CR¹⁰ | H | H | OCF₃ | H | 4.49^{a)} |
| 18 | F | F | H | O | CR¹⁰ | Cl | H | Cl | Cl | |
| 19 | F | F | H | O | CR¹⁰ | H | H | —CH═CH—CH═CH— | | 4.56^{a)} |
| 20 | F | F | H | O | CR¹⁰ | Cl | H | CF₃ | H | 5.41^{a)} |
| 21 | F | F | H | O | CR¹⁰ | H | Me | Cl | Me | 5.81^{a)} |
| 22 | F | F | F | O | CR¹⁰ | H | H | CF₃ | H | 5.31^{a)} |
| 23 | F | F | H | O | CR¹⁰ | H | H | phenyl | H | 4.86^{a)} |
| 24 | F | F | H | O | CR¹⁰ | H | SO₂NHMe | Cl | H | 2.87^{a)} |
| 25 | F | F | F | O | CR¹⁰ | H | H | Cl | H | 5.22^{a)} |
| 26 | F | F | H | O | CR¹⁰ | H | H | Br | H | 4.38^{a)} |
| 27 | F | F | H | S | CR¹⁰ | H | H | CF₃ | H | 4.77^{a)} |
| 28 | F | F | H | O | CR¹⁰ | CF₃ | H | CF₃ | H | 5.31^{a)} |
| 29 | F | F | H | O | CR¹⁰ | H | H | 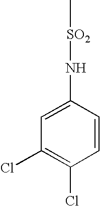 | H | |
| 30 | F | F | H | S | CR¹⁰ | H | H | H | H | |
| 31 | F | F | H | O | CR¹⁰ | H | CO₂Me | H | H | |
| 32 | F | F | H | O | CR¹⁰ | H | —CH═CH—CH═CH— | | H | 4.46^{a)} |
| 33 | F | F | H | O | CR¹⁰ | H | 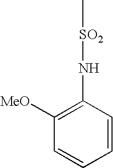 | Cl | H | 4.04^{a)} |
| 34 | F | F | H | O | CR¹⁰ | Cl | —SO₂—CH₂—O— | | H | |
| 35 | F | F | H | O | CR¹⁰ | H | H | CH₂SO₂Me | H | |
| 36 | F | F | F | O | CR¹⁰ | H | H | phenyl | H | 5.72^{a)} |

Preparation of starting materials of the formula (II)

### EXAMPLE II-1

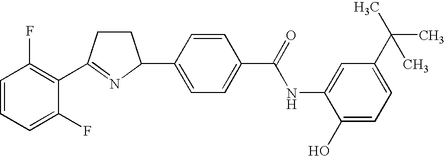

Under argon, a solution of 2.62 g of triphenylphosphine in toluene is added to 492 mg of palladium acetate in 50 ml of toluene. A solution of 3.03 g of 2-(4-bromophenyl)-5-(2,6-difluorophenyl)-3,4-dihydro-2H-pyrrole, 2.97 g of 2-hydroxy-5-tert-butylaniline and 2 g of Hünig base in toluene is then added, and the mixture is allowed to react at a carbon monoxide pressure of 5 bar and at 100° C. for 24 h. After cooling, the mixture is diluted with ethyl acetate and extracted with water and the organic phase is dried over sodium sulphate, filtered and concentrated. The crude product is purified by silica gel chromatography (mobile phase: ethyl acetate/cyclohexane 1:3→1:1).

This gives 2.1 g (52% of theory) of N-(5-tert-btyl-2-hydroxyphenyl)-4-[5-(2,6-difluorohenyl)-3,4-dihydro-2H-pyrrol-2-yl]benzamide.

HPLC: log P (2.3)=3.22

### EXAMPLE II-2

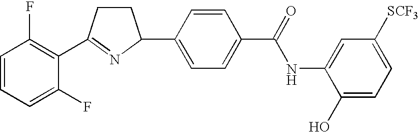

Under argon, 90 mg of palladium acetate are dissolved in 5 ml of toluene. 550 mg of triphenylphosphine in 10 ml of toluene, a solution of 900 mg of 2-amino-5-[(trifluoromethyl)sulphanyl]phenol and 1.38 g of 2-(4-bromophenyl)-5-(2,6-difluoro-phenyl)-3,4-dihydro-2H-pyrrole and 0.8 g of DBU in 5 ml of toluene are added successively. The mixture is stirred at 95° C. under carbon monoxide for 16 h. After cooling, the reaction mixture is concentrated, citric acid, aqueous sodium chloride solution and 2N aqueous sodium hydroxide solution are added, and the reaction mixture is, at pH 3, extracted twice with ethyl acetate. The organic phase is dried, filtered and concentrated. The crude product is purified by silica gel chromatography (mobile phase: cyclohexane→cyclohexane/ethyl acetate 1:1).

This gives 0.5 g (24% of theory) of 4-[5-(2,6-difluorophenyl)-3,4-dihydro-2H-pyrrol-2-yl]-N-{2-hydroxy-5-[(trifluoromethyl)sulphanyl]phenyl}benzamide.

LC-MS: 493.0 [M+H]+

### EXAMPLE II-3

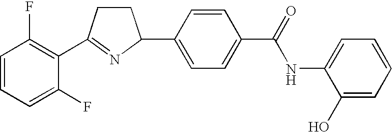

Under argon, 240 mg of palladium acetate are dissolved in 5 ml of toluene. 2.9 g of triphenylphosphine in 10 ml of toluene, a solution of 2 g of 2-aminophenol and 2.95 g of 2-(4-bromophenyl)-5-(2,6-difluorophenyl)-3,4-dihydro-2H-pyrrole and 2 g of Hünig base in 5 ml of toluene are added successively. The mixture is stirred at 95° C. under carbon monoxide for 16 h. After cooling, the reaction mixture is concentrated, citric acid and aqueous sodium chloride solution and 2 N aqueous sodium hydroxide solution are added, and the reaction mixture is, at pH 3, extracted twice with ethyl acetate. The organic phase is dried, filtered and concentrated. The crude product is purified by silica gel chromatography (mobile phase: cyclohexane→cyclohexane/ethyl acetate 1:1).

This gives 3.1 g (90% of theory) of 4-[5-(2,6-difluorophenyl)-3,4-dihydro-2H-pyrrol-2-yl]-N-(2-hydroxyphenyl)benzamide.

¹H-NMR (CD₃CN): δ=8.9 (br, 1H), 8.55 (br, 1H), 7.95 (br, 2H), 7.5 (m, 4H), 7.1 (m, 3H), 6.95 (m, 2H), 5.35 (t, 1H), 3.1 (m, 2H), 2.65 (m, 1H), 1.85 (m, 1H) ppm.

### EXAMPLE II-4

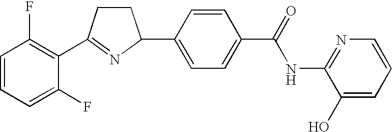

Under argon, a solution of 2.62 g of triphenylphosphine in toluene is added to 492 mg of palladium acetate in 50 ml of toluene. A solution of 3.02 g of 2-(4-bromophenyl)-5-(2,6-difluorophenyl)-3,4-dihydro-2H-pyrrole, 1.98 g of 2-amino-3-hydroxypyridine and 2 g of Hünig base in toluene is then added, a carbon monoxide pressure of 5 bar is applied and the mixture is allowed to react for 24 h. After cooling, the mixture is diluted with ethyl acetate and extracted with water and the organic phase is dried over sodium sulphate, filtered and concentrated. The crude product is purified by silica gel chromatography (mobile phase: cyclohexane/ethyl acetate 1:1).

This gives 2 g (56% of theory) of 4-[5-(2,6-difluorophenyl)-3,4-dihydro-2H-pyrrol-2-yl]-N-(3-hydroxy-2-pyridinyl)benzamide.

HPLC: log P (2.3)=1.16

### EXAMPLE II-5

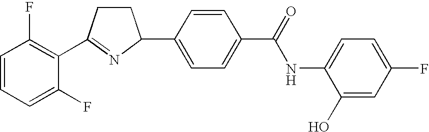

Under argon, 995 mg of triphenylphosphine are dissolved in 15 ml of toluene. A solution of 156 mg of palladium acetate in 5 ml of toluene is added. After 10 min, a solution of 0.89 g of 2-(4-bromophenyl)-5-(2,6-difluorophenyl)-3,4-dihydro-2H-pyrrole, 0.35 g of 2-hydroxy-4-fluoroaniline and 0.5 g of DBU in toluene is added, the atmosphere is changed to carbon monoxide and the mixture is heated at 95° C. for about 20 h. Aqueous citric acid and sodium chloride are then added, and the mixture is extracted with ethyl acetate. The organic phase is dried over sodium sulphate, filtered and concentrated. The crude product is purified by silica gel chromatography (mobile phase: ethyl acetate/cyclohexane 1:1).

This gives 0.7 g (64% of theory) of 4-[5-(2,6-difluorophenyl)-3,4-dihydro-2H-pyrrol-2-yl]-N-(5-fluoro-2-hydroxyphenyl)benzamide.

LC-MS: 411.0 [M+H]+

The log P values given in the Tables and Preparation Examples above are determined in accordance with EEC Directive 79/831 Annex V.A8 by HPLC (High Performance Liquid Chromatography) using a reversed-phase column (C 18). Temperature: 43° C.

The determination in the acidic range is carried out at pH 2.3 using the mobile phases 0.1% aqueous phosphoric acid and acetonitrile; linear gradient from 10% acetonitrile to 90% acetonitrile. In the Tables, the values are marked a).

The determination in the neutral range is carried out at pH 7.5 using the mobile phases 0.01 molar aqueous phosphate buffer solution and acetonitrile; linear gradient 10% acetonitrile to 90% acetonitrile. In the Tables, the values are marked b).

Calibration is carried out using unbranched alkan-2-ones (of 3 to 16 carbon atoms) with known log P values (determination of the log P values by retention times using linear interpolation between two successive alkanones).

The lambda max values were determined in the maxima of the chromatographic signals using the UV spectra from 200 nm to 400 nm.

### USE EXAMPLES

### EXAMPLE A

Heliothis Armigera Test

Solvent: 7 parts by weight of dimethylformamide

Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Soya bean shoots (Glycine max) are treated by being dipped into the preparation of active compound of the desired concentration and are populated with Heliothis armigera caterpillars while the leaves are still moist.

After the desired period of time, the kill in % is determined. 100% means that all caterpillars have been killed; 0% means that none of the caterpillars have been killed.

In this test, for example, the following compounds of the Preparation Examples show good activity:

TABLE A
Plant-damaging insects
Heliothis armigera test

| Active compounds | Concentration of active compound in ppm | Efficacy in % after 7 d |
|---|---|---|
| 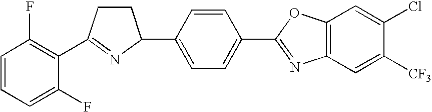 | 100 | 100 |
| 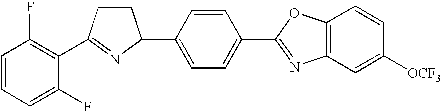 | 100 | 100 |
| 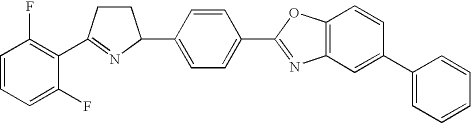 | 100 | 100 |
| 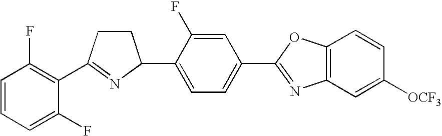 | 100 | 100 |
| 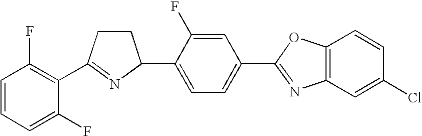 | 100 | 100 |
| 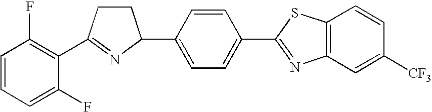 | 100 | 100 |
| 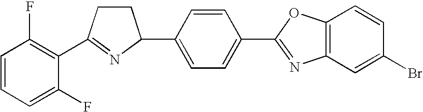 | 100 | 100 |

### EXAMPLE B

Meloidogyne test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

Containers are filled with sand, solution of active compound, Meloidogyne incognita egg/larvae suspension and lettuce seeds. The lettuce seeds germinate and the plants develop. On the roots, galls are formed.

After the desired period of time, the nematicidal action is determined in % by gall formation. 100% means that no galls were found; 0% means that the number of galls on the treated plants corresponds to that of the untreated control.

In this test, for example, the following compounds of the Preparation Examples show good activity:

TABLE B
Plant-damaging insects
Meloidogyne test

| Active compound | Concentration of active compound in ppm | Efficacy in % after 7 d |
|---|---|---|
| 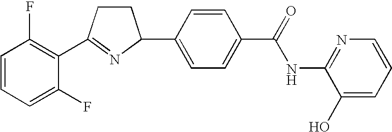 | 20 | 95 |
| 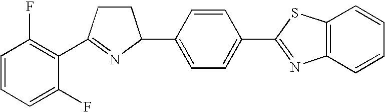 | 20 | 80 |
| 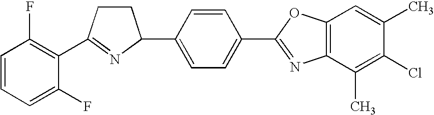 | 20 | 98 |
| 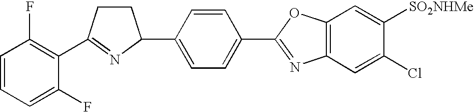 | 20 | 80 |
| 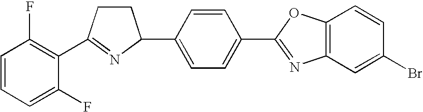 | 20 | 80 |

### EXAMPLE C

Phaedon Larvae Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with larvae of the mustard beetle (Phaedon cochleariae) while the leaves are still moist.

After the desired period of time, the kill in % is determined. 100% means that all beetle larvae have been killed; 0% means that none of the beetle larvae have been killed.

In this test, for example, the following compounds of the Preparation Examples show good activity:

TABLE C
Plant-damaging insects
Phaedon larvae test

| Active compounds | Concentration of active compound in ppm | Efficacy in % after 7 d |
|---|---|---|
| 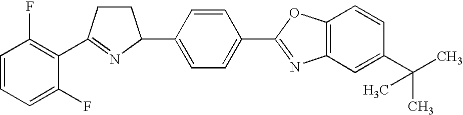 | 500 | 100 |
| 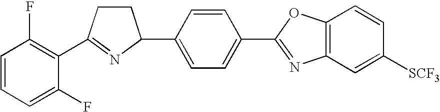 | 500 | 100 |
| 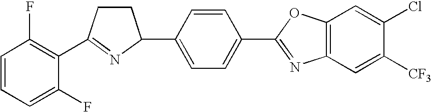 | 100 | 100 |
| 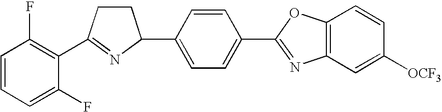 | 100 | 100 |
| 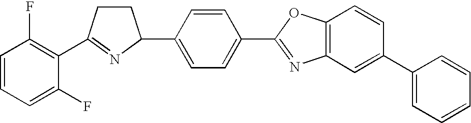 | 100 | 100 |
| 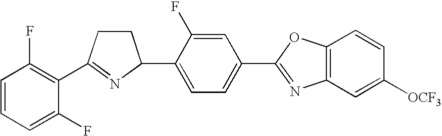 | 100 | 100 |
| 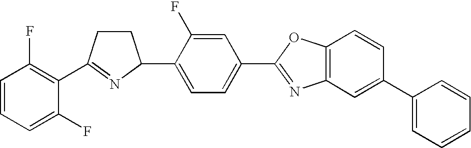 | 100 | 100 |

### EXAMPLE D

Plutella Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the diamondback moth (Plutella xylostella) while the leaves are still moist.

After the desired period of time, the kill in % is determined. 100% means that all caterpillars have been killed; 0% means that none of the caterpillars have been killed.

In this test, for example, the following compound of the Preparation Examples show good activity:

TABLE D
Plant-damaging insects
Plutella test

| Active compounds | Concentration of active compound in ppm | Efficacy in % after 7 d |
|---|---|---|
| 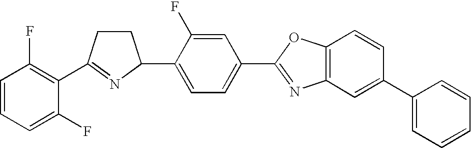 | 100 | 100 |

### EXAMPLE E

Spodoptera Exigua Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the armyworm (Spodoptera exigua) while the leaves are still moist.

After the desired period of time, the kill in % is determined. 100% means that all caterpillars have been killed; 0% means that none of the caterpillars have been killed.

In this test, for example, the following compound of the Preparation Examples show good activity:

TABLE E
Plant-damaging insects
Spodoptera exigua test

| Active compounds | Concentration of active compound in ppm | Efficacy in % after 7 d |
|---|---|---|
| 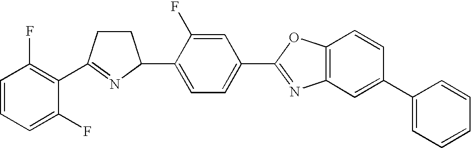 | 100 | 100 |

### EXAMPLE F

Spodoptera Frugiperda Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the armyworm (Spodoptera frugiperda) while the leaves are still moist.

After the desired period of time, the kill in % is determined. 100% means that all caterpillars have been killed; 0% means that none of the caterpillars have been killed.

In this test, for example, the following compounds of the Preparation Examples show good activity:

TABLE F
Plant-damaging insects
Spodoptera frugiperda test

| | Concentration | |
|---|---|---|
| Active compounds | of active compound in ppm | Efficacy in % after 7 d |
| 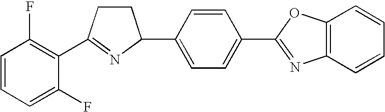 | 500 | 100 |
| 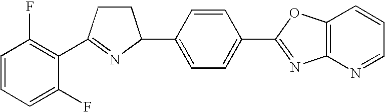 | 500 | 100 |
| 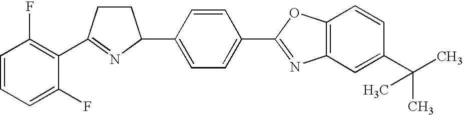 | 500 | 100 |
| 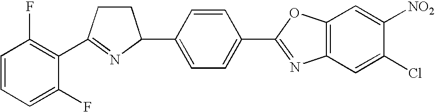 | 500 | 100 |
| 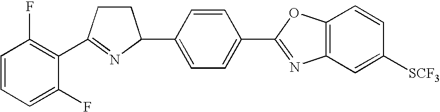 | 500 | 100 |
| 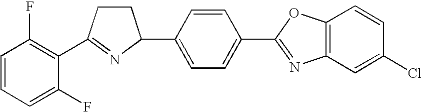 | 100 | 100 |
| 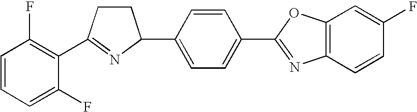 | 500 | 100 |
| 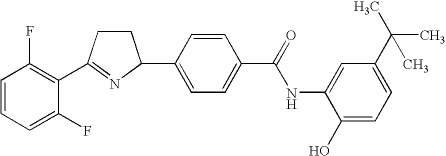 | 500 | 100 |
| 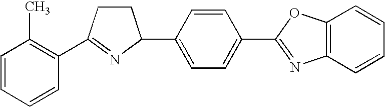 | 100 | 100 |
| 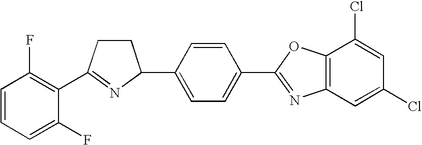 | 100 | 100 |
| 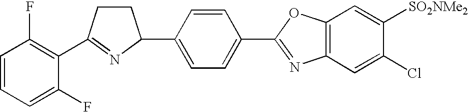 | 100 | 100 |
| 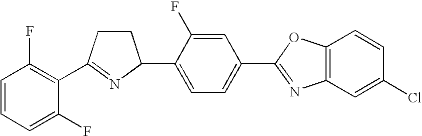 | 100 | 100 |
| 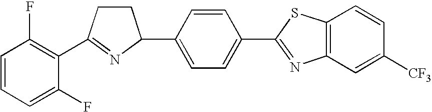 | 100 | 100 |
| 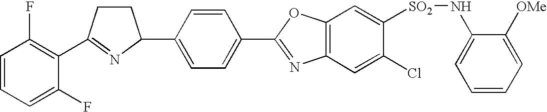 | 500 | 100 |

### EXAMPLE G

Tetranychus Test (OP-Resistant/Dip Treatment)

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Bean plants (Phaseolus vulgaris) which are heavily infested by all stages of the greenhouse red spider mite (Tetranychus urticae) are treated by being dipped into a preparation of active compound of the desired, concentration.

After the desired period of time, the effect in % is determined. 100% means that all spider mites have been killed; 0% means that none of the spider mites have been killed.

In this test, for example, the following compounds of the Preparation Examples show good activity:

TABLE G
Plant-damaging insects
Tetranychus test (OP-resistant/dip treatment)

| Active compound | Concentration of active compound in ppm | Efficacy in % after 7 d |
|---|---|---|
| 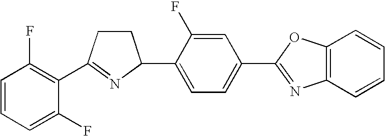 | 100 | 95 |
| 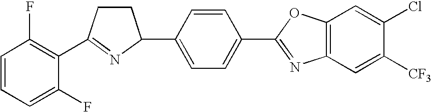 | 100 | 98 |
| 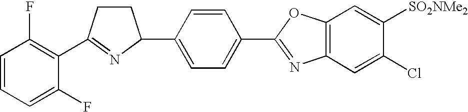 | 100 | 90 |
| 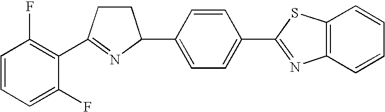 | 100 | 99 |
| 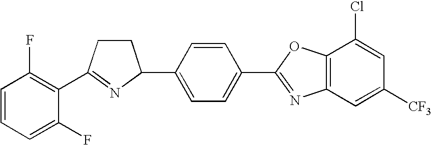 | 100 | 95 |
| 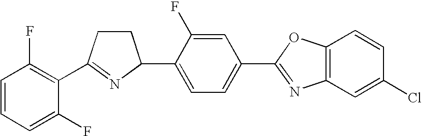 | 100 | 95 |
| 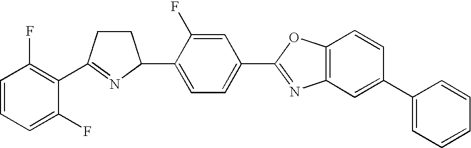 | 100 | 99 |
| 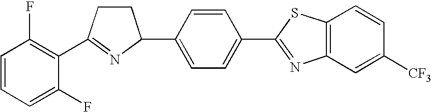 | 100 | 90 |
| 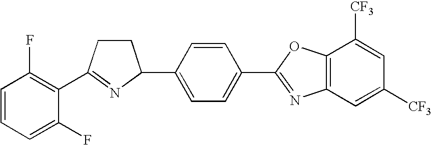 | 100 | 100 |

### EXAMPLE H

Diabrotica Balteata Test (Larvae in Soil)

Critical concentration test/soil insects—Treatment of transgenic plants

| | |
|---|---|
| Solvent: | 7 parts by weight of dimethylformamide |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

The preparation of active compound is poured onto the soil. The concentration of active compound in the preparation is virtually immaterial, only the amount by weight of active compound per volume unit of soil, which is stated in ppm (mg/l) matters. The soil is filled into 0.25 1 pots, and these are allowed to stand at 20° C.

Immediately after the preparation, 5 pregerminated maize corns of the cultivar YIELD GUARD (trademark of Monsanto Comp., USA) are placed into each pot. After 2 days, the corresponding test insects are placed into the treated soil. After a further 7 days, the efficacy of the active compound is determined by counting the number of maize plants that have emerged (1 plant=20% activity).

### EXAMPLE I

Heliothis Virescens Test (Treatment of Transgenic Plants)

| | |
|---|---|
| Solvent: | 7 parts by weight of dimethylformamide |
| Emulsifier: | 1 part by weight of alkylaryl polyglycol ether |

To produce a suitable preparation of active compound, 1 part by weight of active compound is mixed with the stated amount of solvent and the stated amount of emulsifier, and the concentrate is diluted with water to the desired concentration.

Soya bean shoots (Glycine max) of the cultivar Roundup Ready (trademark of Monsanto Comp. USA) are treated by being dipped into the preparation of active compound of the desired concentration and are populated with the tobacco budworn caterpillar Heliothis virescens while the leaves are still moist.

After the desired period of time, the kill in % is determined. 100% means that all caterpillars have been killed; 0% means that none of the caterpillars have been killed.

1-18. (canceled)

19. A Δ¹-pyrroline of formula (I) 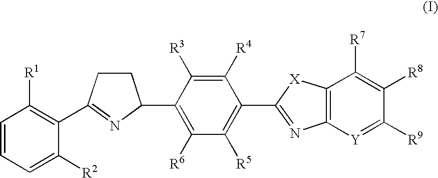
in which
R¹ represents halogen or methyl,
R² represents hydrogen or halogen,
R³, R⁴, R⁵, and R⁶ independently of one another represent hydrogen, halogen, C₁-C₄-alkyl, or C₁-C₄-alkoxy,
X represents O or S,
Y represents CR¹⁰ or N,
R⁷, R⁸, R⁹, and R¹⁰ independently of one another represent hydrogen, halogen, nitro, C₁-C₄-alkyl, C₁-C₄-haloalkyl, C₁-C₄-haloalkoxy, C₁-C₄-haloalkylthio, C₁-C₄-alkoxycarbonyl, phenyl, —(CH₂)ₘ—SO₂R¹¹, or —SO₂NR¹²R¹³,
or R⁷ and R⁸ together or R⁸ and R⁹ together or R⁹ and R¹⁰ together form a saturated or unsaturated 5- or 6-membered ring that optionally contains one or two heteroatom groupings selected from the group consisting of N, O, S, and SO₂,
m represents 0 or 1,
R¹¹ represents C₁-C₄-alkyl or morpholino,
R¹² represents C₁-C₄-alkyl or represents phenyl that is optionally mono- to tetrasubstituted by identical or different substituents selected from the group consisting of halogen, C₁-C₄-alkyl, and C₁-C₄-alkoxy, and
R¹³ represents hydrogen or C₁-C₄-alkyl.

20. A Δ¹-pyrroline of formula (I) according to claim 19 in which
R¹ represents fluorine, chlorine, bromine, or methyl,
R² represents hydrogen, fluorine, or chlorine,
R³, R⁴, R⁵, and R⁶ independently of one another represent hydrogen, fluorine, chlorine, bromine, C₁-C₄-alkyl, or C₁-C₄-alkoxy,
X represents O or S,
Y represents CR¹⁰ or N,
R⁷, R⁸, R⁹, and R¹⁰ independently of one another represent hydrogen, fluorine, chlorine, bromine, nitro, C₁-C₄-alkyl; represent C₁-C₄-haloalkyl, C₁-C₄-halo-alkoxy, or C₁-C₄-haloalkylthio having in each case 1 to 9 fluorine, chlorine, and/or bromine atoms; or represent C₁-C₄-alkoxycarbonyl, phenyl, —(CH₂)ₘ—SO₂R¹¹, or —SO₂NR¹²R¹³,
or R⁷ and R⁸ together or R⁸ and R⁹ together or R⁹ and R¹⁰ together form a saturated or unsaturated 5- or 6-membered ring that optionally contains one or two heteroatom groupings selected from the group consisting of N, O, S, and SO₂,
m represents 0 or 1,
R¹¹ represents C₁-C₄-alkyl or morpholino,
R¹² represents C₁-C₄-alkyl or represents phenyl that is optionally mono- to tetrasubstituted by identical or different substituents selected from the group consisting of fluorine, chlorine, bromine, C₁-C₄-alkyl, and C₁-C₄-alkoxy, and R¹³ represents hydrogen or C₁-C₄-alkyl.

21. A Δ¹-pyrroline of formula (I) according to claim 19 in which
R¹ represents fluorine, chlorine, or methyl,
R² represents hydrogen, fluorine, or chlorine,
R³ and R⁶ independently of one another represent hydrogen, fluorine, chlorine, methyl, ethyl, n-propyl, i-propyl, n-butyl, i-butyl, s-butyl, t-butyl, methoxy, or ethoxy,
R⁴ and R⁵ each represent hydrogen,
X represents O or S,
Y represents CR¹⁰ or N,
R⁷, R⁸, R⁹, and R¹⁰ independently of one another represent hydrogen, fluorine, chlorine, bromine, nitro, methyl, ethyl, n-propyl, i-propyl, n-butyl, i-butyl, s-butyl, t-butyl; represent C₁-C₄-haloalkyl, C₁-C₄-haloalkoxy, or C₁-C₄-halo-alkylthio having in each case 1 to 9 fluorine, chlorine, and/or bromine atoms; or represent methoxycarbonyl, ethoxycarbonyl, phenyl, —(CH₂)ₘ—SO₂R¹¹ or —SO₂NR¹²R¹³,
or R⁷ and R⁸ together or R⁸ and R⁹ together or R⁹ and R¹⁰ together form a saturated or unsaturated 5- or 6-membered ring that optionally contains one or two heteroatom groupings selected from the group consisting of N, O, S, and SO₂,
m represents 0 or 1,
R¹¹ represents methyl, ethyl, n-propyl, i-propyl, n-butyl, i-butyl, s-butyl, t-butyl, or morpholino,
R¹² represents methyl, ethyl, n-propyl, i-propyl, n-butyl, i-butyl, s-butyl, or t-butyl or represents phenyl that is optionally mono- to trisubstituted by identical or different substituents selected from the group consisting of fluorine, chlorine, bromine, methyl, ethyl, n-propyl, i-propyl, n-butyl, i-butyl, s-butyl, t-butyl, methoxy, ethoxy, n-propoxy, i-propoxy, n-butoxy, i-butoxy, s-butoxy, and t-butoxy, and
R¹³ represents hydrogen, methyl, ethyl, n-propyl, i-propyl, n-butyl, i-butyl, s-butyl, or t-butyl.

22. A Δ¹-pyrroline of formula (I) according to claim 19 in which
R¹ represents fluorine, chlorine, or methyl,
R² represents hydrogen, fluorine, or chlorine,
R³ represents hydrogen, fluorine, chlorine, methyl, methoxy, or ethoxy,
R⁴, R⁵, and R⁶ each represent hydrogen,
X represents O,
Y represents CR¹⁰ or N, and
R⁷, R⁸, R⁹, and R¹⁰ independently of one another represent hydrogen, fluorine, chlorine, bromine, nitro, methyl, i-propyl, t-butyl, trifluoromethyl, trifluoromethoxy, trifluoromethylthio, methoxycarbonyl, phenyl, —SO₂-morpholino, —CH₂SO₂Me, —SO₂NHMe, —SO₂NMe₂, —SO₂NH-(3,4-dichlorophenyl), or —SO₂NH-(2-methoxyphenyl).

23. A Δ¹-pyrroline of formula (I) according to claim 19 in which R¹ and R² represent fluorine.

24. A Δ¹-pyrroline of formula (I) according to claim 19 in which Y represents CR¹⁰.

25. A Δ¹-pyrroline of formula (I) according to claim 24 in which R¹⁰ represents hydrogen.

26. A Δ¹-pyrroline of formula (I) according to claim 19 in which R³ represents hydrogen or fluorine.

27. A Δ¹-pyrroline of formula (I) according to claim 19 in which X represents O.

28. A Δ¹-pyrroline of formula (I) according to claim 19 in which X represents S.

29. A Δ¹-pyrroline of formula (I) according to claim 19 in which R⁴ and R⁵ represent hydrogen.

30. A Δ¹-pyrroline of formula (I) according to claim 19 in which R¹ and R² represent fluorine, X represents O, and Y represents CH.

31. An (R)-configured compound of formula (I-a) 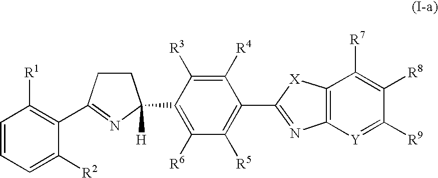
R¹ represents halogen or methyl,
R² represents hydrogen or halogen,
R³, R⁴, R⁵, and R⁶ independently of one another represent hydrogen, halogen, C₁-C₄-alkyl, or C₁-C₄-alkoxy,
X represents O or S,
Y represents CR¹⁰ or N,
R⁷, R⁸, R⁹, and R¹⁰ independently of one another represent hydrogen, halogen, nitro, C₁-C₄-alkyl, C₁-C₄-haloalkyl, C₁-C₄-haloalkoxy, C₁-C₄-haloalkylthio, C₁-C₄-alkoxycarbonyl, phenyl, —(CH₂)ₘ—SO₂R¹¹, or —SO₂NR¹²R¹³,
or R⁷ and R⁸ together or R⁸ and R⁹ together or R⁹ and R¹⁰ together form a saturated or unsaturated 5- or 6-membered ring that optionally contains one or two heteroatom groupings selected from the group consisting of N, O, S, and SO₂,
m represents 0 or 1,
R¹¹ represents C₁-C₄-alkyl or morpholino,
R¹² represents C₁-C₄-alkyl or represents phenyl that is optionally mono- to tetrasubstituted by identical or different substituents selected from the group consisting of halogen, C₁-C₄-alkyl, and C₁-C₄-alkoxy, and
R¹³ represents hydrogen or C₁-C₄-alkyl.

32. A process for preparing a compound of formula (I) according to claim 19 comprising reacting a compound of formula (II) 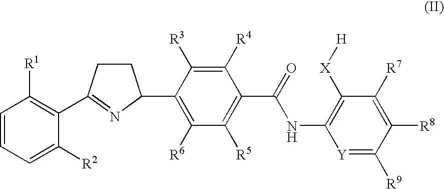
in which R¹, R², R³, R⁴, R⁵, R⁶, X, Y, R⁷, R⁸, and R⁹ are as defined for formula (I) of claim 19,
with p-toluenesulphonic acid,
optionally in the presence of a diluent.

33. A pesticide comprising one or more compounds of formula (I) according to claim 19 and one or more extenders and/or surfactants.

34. A method for controlling pests comprising allowing an effective amount of a compound of formula (I) according to claim 19 to act on pests and/or their habitat.

35. A process for preparing a pesticides comprising mixing a compound of formula (I) according to claim 19 with one or more extenders and/or surfactants.

36. A compound of formula (II) 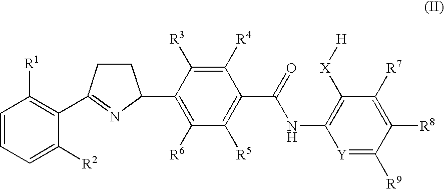
in which
R¹ represents halogen or methyl,
R² represents hydrogen or halogen,
R³, R⁴, R⁵, and R⁶ independently of one another represent hydrogen, halogen, C₁-C₄-alkyl, or C₁-C₄-alkoxy,
X represents O or S,
Y represents CR¹⁰ or N,
R⁷, R⁸, R⁹, and R¹⁰ independently of one another represent hydrogen, halogen, nitro, C₁-C₄-alkyl, C₁-C₄-haloalkyl, C₁-C₄-haloalkoxy, C₁-C₄-haloalkylthio, C₁-C₄-alkoxycarbonyl, phenyl, —(CH₂)ₘ—SO₂R¹¹, or —SO₂NR¹²R¹³,
or R⁷ and R⁸ together or R⁸ and R⁹ together or R⁹ and R¹⁰ together form a saturated or unsaturated 5- or 6-membered ring that optionally contains one or two heteroatom groupings selected from the group consisting of N, O, S, and SO₂,
m represents 0 or 1,
R¹¹ represents C₁-C₄-alkyl or morpholino,
R¹² represents C₁-C₄-alkyl or represents phenyl that is optionally mono- to tetrasubstituted by identical or different substituents selected from the group consisting of halogen, C₁-C₄-alkyl, and C₁-C₄-alkoxy, and
R¹³ represents hydrogen or C₁-C₄-alkyl.
